(19) United States
(12) Patent Application Publication
(10) Pub. No.: US 2024/0108910 A1
(43) Pub. Date: Apr. 4, 2024

(54) THORACIC SPINAL NERVE NEUROMODULATION THERAPY FOR GASTROINTESTINAL SYMPTOMS

(71) Applicant: Augusta University Research Institute, Inc., Augusta, GA (US)

(72) Inventor: Amol Sharma, Evans, GA (US)

(21) Appl. No.: 18/479,645

(22) Filed: Oct. 2, 2023

Related U.S. Application Data
(60) Provisional application No. 63/377,933, filed on Sep. 30, 2022.

(51) Int. Cl. A61N 2/00; A61N 2/02

(52) U.S. Cl. CPC A61N 2/006; A61N 2/02

(57) ABSTRACT

Systems and methods for neuromodulation therapy with repetitive magnetic stimulation of paraspinal regions have been developed for the treatment and prevention of gastroparesis. The methods identify a minimal electromagnetic intensity threshold required to elicit a motor response of 10 μV at a preferred location within the mid-thoracic region of a subject, and then deliver repetitive magnetic stimulation to the subject at a maximum 150% of the threshold value. Typically, the methods administer about 1,200 stimulations in groups of about 300 pulses having a frequency of about 1 Hz, with a rest period of about 5 minutes between pulses. Typically, the methods repeat the stimuli about twice a day for a period of around 5 days.

### CROSS-REFERENCE TO RELATED APPLICATIONS

This application claims the benefit of and priority to U.S. Provisional Application No. 63/377,933 filed Sep. 30, 2023, which is hereby incorporated by reference in its entirety.

### STATEMENT REGARDING FEDERALLY SPONSORED RESEARCH

This invention was made with Government Support under DK076169 and DK115255 awarded by the National Institute of Diabetes and Digestive and Kidney Diseases (NIDDK). The Government has certain rights in the invention.

### FIELD OF THE INVENTION

The invention is generally related to systems and methods for treatment of neuropathies, particularly to therapeutic neuromodulation of thoracic spinal nerves for the amelioration of gastrointestinal symptoms in patients in need thereof.

### BACKGROUND OF THE INVENTION

Diabetic gastroparesis (DGp) is a serious condition characterized by presence of six cardinal gastrointestinal (GI) symptoms and delayed gastric emptying in the absence of mechanical obstruction. The six cardinal GI symptoms include nausea, vomiting, early satiety, postprandial fullness, and abdominal pain. It is associated with severe symptoms, impaired quality of life, and significant healthcare utilization (Sharma, et al., Current Gastroenterology Reports 2020;22:1-10).

Gastroparesis is a late manifestations of diabetic autonomic neuropathy, which precedes cardiac autonomic neuropathy and associated with silent myocardial infarction and a high 5-year mortality. There are limited effective treatments for diabetic gastroparesis. Abnormal autonomic function test results, indicative of the presence of autonomic neuropathy, can differentiate between DGp subjects and diabetics with normal gastric emptying (Nguyen, et al., Neurogastroenterology & Motility 2020;32:e13810). The autonomic nervous system modulates the stomach and foregut through the parasympathetic innervation by the vagus nerve and sympathetic innervation by the greater splanchnic nerve, originating from thoracic (T5-T10) levels of the spinal cord. In addition to autonomic neuropathy, abnormal sensory signal processing, indicative of afferent visceral neuropathy, is also associated with gastroparesis symptoms (Brock, et al., Diabetes Care 2013;36:3698-3705). Despite having severe gastroparesis symptoms, subjects with DGp have diminished sensation in their GI tract, demonstrated by severe visceral hyposensitivity with increased sensory thresholds to electrical stimulation of the gut, implying a discordance between symptoms and visceral sensation (Brock, et al., Diabetes Care 2013;36:3698-3705).

Treatment of DGp remains unsatisfactory. Metoclopramide is the only FDA-approved medication for gastroparesis. Gastric electrical stimulation is approved for compassionate use in refractory patients, however, it is invasive, lacks robust supporting evidence, does not consistently improve symptoms, and is associated with serious adverse events (Levinthal, et al., Autonomic Neuroscience 2017;202:45-55). The NIH Gastroparesis Registry has followed patients for more than a decade with significant insight. Despite expert management, only 28% of patients had a significant reduction in their symptom scores (Pasricha, et al., Gastroenterology 2015;149:1762-1774. e4). Over 4 years, approximately 40% of patients normalize their gastric emptying without improvement in symptoms (Pasricha, et al., Gastroenterology 2021;160:2006-2017).

There is a large unmet need for therapeutic options to treat refractory GI symptoms, including those of diabetic gastroparesis (DGp). Therefore, it is an object of the invention to provide systems and methods for the effective treatment and management of refractory nausea, vomiting, early satiety, postprandial fullness, and abdominal pain.

It is another object of the invention to provide improved systems and methods to identify and monitor and treat autonomic and visceral neuropathies.

### SUMMARY OF THE INVENTION

Systems and methods for the non-invasive treatment of refractory Gi symptoms have been developed. The methods enhance gastrointestinal (Gi) motility in a subject in need thereof by stimulation of the thoracic spinal sympathetic and/or parasympathetic nerves of the subject. Typically, the methods administer to a subject a stimulus of an intensity between about 100% and about 150%, inclusive, of the minimal intensity required to achieve a motor evoked response (MEP) in the subject (to the extent it is possible to achieve an MEP in the subject). In some forms, the methods further include one or more steps for mapping of the thoracic regions of the subject to identify (i) preferred anatomical location(s) (e.g., including, but not limited to, optimal location) and/or (ii) a minimal intensity of electrical or magnetic stimulation required to achieve a motor evoked response (MEP) in the Gi tract of the subject. In some forms, the mapping identifies a minimal intensity required to evoke an MEP of between about 1 μV and about 50 μV, inclusive, in the Gi tract of the subject. In certain forms, the mapping identifies the anatomical location and intensity required to evoke an MEP of about 10 μV in the Gi tract of the subject.

In some forms, the MEP is evoked in the upper rectus abdominis and/or external oblique muscles of the subject. In some forms, the anatomical location includes the left and right posterior mid-thoracic regions of the subject. Typically, the stimulus is administered to the subject in the form of an electro-magnetic stimulation, for example, as applied by an electromagnetic coil.

Exemplary electromagnetic coils include a single-pulse circular 90 mm coil. Exemplary electromagnetic coils include a 70 mm double air film self-cooling coil. In some forms, the electromagnetic coil is held in place by a coil fixator device throughout administration of electromagnetic stimulation.

In some forms, the administered electromagnetic stimulus includes between about 100 and 100,000 stimulations. For example, in some forms, the methods administer between about 100 and about 100,000 stimulations, inclusive, per anatomical location, e.g., between about 1,000 and about 10,000 stimulations, inclusive, per anatomical location, or between about 10,000 and about 15,000 stimulations, inclusive per anatomical location. In certain forms, the methods administer about 12,000 stimulations per anatomical location. Typically, each electromagnetic stimulus has a frequency of between about 0.1 Hz and about 10 Hz, inclusive, for example, between about 0.5 Hz and about 5 Hz, inclusive. In certain forms, each electromagnetic stimulus has a frequency of between about 1 Hz. In some forms, the stimulations are administered to the subject in groups of pulses. Typically, each group includes between about 10 and about 1,000 pulses, such as between about 100 and 500 pulses. In certain forms, each train includes 300 pulses. Typically, the refractory period between each train of pulses is between about 10 seconds and about 30 minutes, inclusive, such as between about one minute and about ten minutes, inclusive. In certain forms, the refractory period between each train of pulses is about five minutes.

The methods can be carried out once, or multiple times in a regimen of repeating treatments. In some forms, the methods are carried out multiple times on a single subject. In other forms, when the methods include one or more mapping steps, the methods perform the mapping once, then repeat administration of the stimuli once or more than once in the same subject. Typically, the methods are continuously applied to a subject until a desired therapeutic effect is achieved in the subject. In some forms, the methods are administered to a subject once an hour, once every 12 hours, once a day, once every other day, or once a week, once a month, or less frequently than once a month, such as once a year. In certain forms, the methods administer stimuli to a subject two times a day for a period of five consecutive days. Therefore, in some forms, the methods include a treatment regimen of ten rounds of therapy, wherein each round of therapy includes optionally performing a mapping step and administering stimuli to the subject.

In some forms, the methods include administering one or more additional therapeutic agents or procedures to the subject in conjunction with stimuli and optionally mapping of the subject. The additional therapeutic agent and/or procedure can be administered concomitantly, before or after the methods. In some forms, the methods include administering one or more additional therapeutic agents or procedures to the subject during a treatment regimen, e.g., between repeating rounds of treatment with stimuli and optionally mapping of the subject. In other forms, the methods include administering one or more additional therapeutic agents or procedures before the start of a treatment regimen or after the completion of a treatment regimen including two or more rounds of repeating the stimuli and optionally mapping of the subject. In some forms, the additional active agent is a neuromodulator, a prokinetic agent, or an antiemetic agent, or a combination of a neuromodulator and/or a prokinetic and/or antiemetic agent. In certain forms, the additional active agent is Metoclopramide.

In some forms, the methods treat or prevent one or more gastric symptoms in the subject. Exemplary symptoms that can be treated or prevented by the methods include anal sphincter dysfunction, nausea, vomiting, retching, early satiety, postprandial fullness, bloating, loss of appetite, weight loss, abdominal distension and upper abdominal pain. In some forms, the methods treat or prevent one or more chronic symptoms of gastroparesis in the subject. In some forms, the methods are effective to reduce the number of vomiting episodes (emesis) in the subject over a set period of time, e.g., a 24 hour period. In some forms, the methods are effective to treat or prevent one or more symptoms identified in the ANMS Gastroparesis Cardinal Symptom Index (ANMS GCSI-DD). For example, in some forms, the methods are effective to improve one or more of the symptoms identified in the ANMS GCSI-DD by one or more points, wherein the points are defined as 0=none, 1=mild, 2=moderate, 3=severe, and 4=very severe. In certain forms, the methods are effective to improve one or more of the symptoms identified in the ANMS GCSI-DD by reduction of one point, two points, three points, or four points.

In some forms, the methods treat or prevent the one or more symptoms in a subject for a period of one day, one week, two weeks, three weeks, four weeks, one month, two months, three months, four months, five months, six months, or more than six months, such as one year or more. In some forms, the efficacy of the methods is measured by comparison with a suitable control, such as an untreated subject.

Typically, the subject is a symptomatic patient having delayed gastric emptying without evidence of obstruction. Absence of obstruction is determined, for example, by computed tomography (CT), a gastric emptying breath test (GEBT), upper endoscopy, or an upper gastrointestinal radiographic series, which can also assess the small intestine. In some forms, the subject has been diagnosed as having, or is suspected as having idiopathic gastroparesis, diabetic gastroparesis, or surgical gastroparesis. In certain forms, the subject has been diagnosed as having diabetic gastroparesis. In some forms, the subject has not been identified as having severe cardiac disease and arrhythmias, or as having of metal implant or prosthesis.

### BRIEF DESCRIPTION OF THE DRAWINGS

FIG. 1 is a photograph depicting a ThorS-MagNT treatment method performed on a subject, showing ThorS-MagNT treatment administered with [A] a 70-mm double air-film self-cooling coil (MAGSTIM Rapid2, Whitland, UK) placed on marked bilateral posterior thoracic locations, held in place by [B] a coil fixator, and connected to [C] a generator.

FIG. 2 is a flow-diagram for a representative treatment schedule of 10 sessions over 5 days, showing Seven-day ANMS Gastroparesis Cardinal Symptom Index Daily Diary (ANMS GCSI-DD) completed every evening 1 hour after dinner from 7 days prior to treatment to 21 days post-treatment. FDA responder definition is >30% improvement in 7-day total ANMS GCSI-DD.

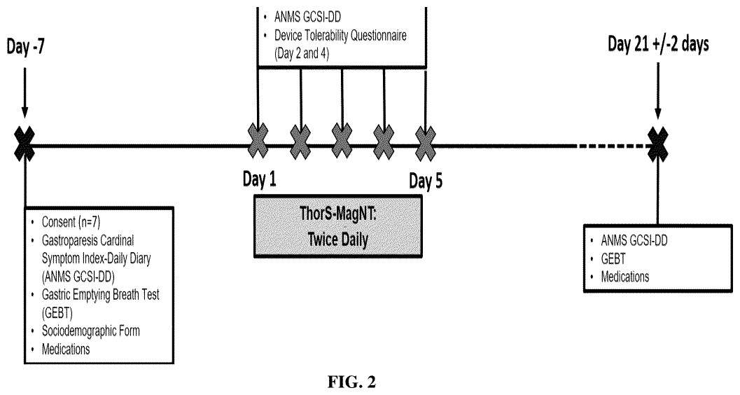

FIG. 3 is a graph showing percent reduction of total ANMS GCSI-DD after ThorS-MagNT from baseline. Subjects (n=4) treated at optimal intensities (150% of their motor thresholds) had 74.9% reduction during treatment week in total ANMS GCSI-DD score from baseline and met FDA-responder definition (greater than 30% reduction in total ANMS GCSI-DD score) in comparison to 10.1% reduction in subjects treated with low intensities (n=3). Symptom improvement persisted 2 weeks post-treatment with an average of 97.7% symptom reduction versus 0.3% reduction in subjects treated with lower intensities.

FIG. 4 is a graph of average percent reduction during treatment week from baseline for each specific gastroparesis cardinal symptom of the ANMS GCSI-DD and number of vomiting episodes in responders to ThorS-MagNT (n=4).

### DETAILED DESCRIPTION OF THE INVENTION

### I. Definitions

The term “subject” means any individual who is the target of diagnosis or treatment administration. The subject can be a vertebrate, for example, a mammal. Thus, the subject can be a human. The term does not denote a particular age or sex. A patient refers to a subject afflicted with a disease or disorder. The term “patient” includes human and veterinary subjects.

The term “therapeutically effective” means that the amount of a procedure or composition used is of sufficient quantity to ameliorate one or more causes or symptoms of a disease or disorder. Such amelioration only requires a reduction or alteration, not necessarily elimination.

By “treat” or “treatment” is meant the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

“Inhibit,” “inhibiting,” and “inhibition” mean to decrease an activity, response, condition, disease, or other biological parameter. This can include but is not limited to the complete ablation of the activity, response, condition, or disease. This may also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels.

Use of the term “about” is intended to describe values either above or below the stated value in a range of approx. +/−10%; in other forms the values may range in value either above or below the stated value in a range of approx. +/−5%; in other forms the values may range in value either above or below the stated value in a range of approx. +/−2%; in other forms the values may range in value either above or below the stated value in a range of approx. +/−1%. The preceding ranges are intended to be made clear by context, and no further limitation is implied.

The use of any and all examples, or exemplary language (e.g., “such as”) provided herein, is intended merely to better illuminate the description and does not pose a limitation on the scope of the description unless otherwise claimed.

Disclosed are materials, compositions, and components that can be used for, can be used in conjunction with, can be used in preparation for, or are products of the disclosed method and compositions. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a ligand is disclosed and discussed and a number of modifications that can be made to a number of molecules including the ligand are discussed, each and every combination and permutation of ligand and the modifications that are possible are specifically contemplated unless specifically indicated to the contrary. Thus, if a class of molecules A, B, and C are disclosed as well as a class of molecules D, E, and F and an example of a combination molecule, A-D is disclosed, then even if each is not individually recited, each is individually and collectively contemplated. Thus, in this example, each of the combinations A-E, A-F, B-D, B-E, B-F, C-D, C-E, and C-F are specifically contemplated and should be considered disclosed from disclosure of A, B, and C; D, E, and F; and the example combination A-D. Likewise, any subset or combination of these is also specifically contemplated and disclosed. Thus, for example, the sub-group of A-E, B-F, and C-E are specifically contemplated and should be considered disclosed from disclosure of A, B, and C; D, E, and F; and the example combination A-D. Further, each of the materials, compositions, components, etc. contemplated and disclosed as above can also be specifically and independently included or excluded from any group, subgroup, list, set, etc. of such materials.

These concepts apply to all aspects of this application including, but not limited to, steps in methods of making and using the disclosed compositions. Thus, if there are a variety of additional steps that can be performed it is understood that each of these additional steps can be performed with any specific form or combination of forms of the disclosed methods, and that each such combination is specifically contemplated and should be considered disclosed. All methods described herein can be performed in any suitable order unless otherwise indicated or otherwise clearly contradicted by context. The use of any and all examples, or exemplary language (e.g., “such as”) provided herein, is intended merely to better illuminate the embodiments and does not pose a limitation on the scope of the embodiments unless otherwise claimed. No language in the specification should be construed as indicating any non-claimed element as essential to the practice of the invention.

### II. Methods to Increase Gastrointestinal Motility

Methods of using electromagnetic stimulation for treating or preventing one or more refractory GI symptoms have been developed.

Methods of Thoracic Spinal Nerve Magnetic Neuromodulation Therapy (ThorS-MagNT) administer electromagnetic stimuli to the thoracic regions of a subject in an amount effective to increase gastrointestinal motility in the subject. The methods treat and prevent refractory GI symptoms in a subject, including symptoms associated with a gastric (e.g., foregut, midgut, hindgut, liver, or pancreatic) disease or disorder. Exemplary diseases and disorders include diabetic gastroparesis, diabetic autonomic neuropathy, efferent brain-gut neuropathy and afferent gut-brain neuropathy.

Typically, the methods administer an electromagnetic stimulus to the thoracic region (e.g., upper rectus abdominis and/or external oblique muscles) of the subject. The electromagnetic stimulus generally includes an intensity between 100% and 150% of the minimal intensity of electromagnetic stimulation required to achieve a motor evoked response (MEP) of 10 μV in the upper rectus abdominis and/or external oblique muscles of the subject, which may be limited by body habitus. If MEPs are unable to be obtained, a graded stimulus approach can be used based on body mass index (BMI).

In some forms, the subject is diagnosed as having, or as being at increased risk of having a chronic or acute gastric disease or disorder associated with reduced gastrointestinal motility, such as diabetic gastroparesis, diabetic autonomic neuropathy, efferent brain-gut neuropathy and afferent gut-brain neuropathy.

In some forms, the methods include one or more steps to determine the preferred electromagnetic stimulation for stimulating the thoracic sympathetic and parasympathetic nerves and/or vagus nerve of the subject to stimulate and improve gastrointestinal motility in the subject. The anatomical location and the intensity of electromagnetic stimulation needed to provide preferred therapeutic neuromodulation is typically patient-specific and can vary from one patient to another. Therefore, in some forms the methods include a mapping procedure to determine the preferred anatomical location for electromagnetic stimulation and to identify the resting motor threshold for each subject. The methods then apply the information obtained by the mapping procedure to determine the intensity of electromagnetic stimulation required for preferred neuromodulation. Typically, the preferred intensity is between about 100% and about 150%, inclusive, of the threshold value identified in the mapping procedure.

For example, methods for treatment of refractory GI symptoms in a subject including electromagnetic stimulation of the thoracic sympathetic and/or parasympathetic nerves of the subject can include optionally mapping of the thoracic regions of the subject, whereby the mapping includes identifying an anatomical location and minimal intensity of electromagnetic stimulation required to achieve a motor evoked response (MEP) value in the gastrointestinal (GI) tract of the subject; and
administering to a preferred anatomical location electromagnetic stimuli having an intensity between about 100% and about 150%, inclusive, of the minimal intensity of electromagnetic stimulation required to achieve a motor evoked response (MEP) value in the gastrointestinal (GI) tract of the subject.

In some forms, (i) is present and (ii) includes administering to the location identified in (i) an electromagnetic stimulus having an intensity between 100% and about 150% of the minimal electromagnetic intensity identified in (i).

As demonstrated in the Examples, it has been established that an electromagnetic stimuli including about 1,200 pulses and having a frequency of 1 Hz is effective when delivered in groups of 300 pulses separated by a rest period of 5 minutes. Typically, the subject is a human.

### A. Mapping Effective Neurostimulation in the Thoracic Regions

In some forms, the ThorS-MagNT methods optionally include one or more mapping steps to determine the preferred and optimal anatomical location(s) in the thoracic regions of a subject to deliver electromagnetic stimulation for enhancing gastric contractility. An optimal anatomical location(s) is/are the most preferred anatomical location(s), e.g., as identified by mapping, or through other means.

In some forms, the methods examine one or more locations on the skin of the thoracic regions of a subject to administer electromagnetic stimulation into the body of the subject. The electromagnetic stimuli are typically applied to the skin of the subject, e.g., by placing an electromagnetic generator/actuator device (such as an electromagnetic coil) onto the surface of the skin in the thoracic region of the subject. In some forms, the electromagnetic stimuli are administered to the back of the subject, for example, by placing an electromagnetic generator/actuator device (such as an electromagnetic coil) directly onto the skin on the back of the subject in the thoracic region. In exemplary forms, the electromagnetic stimuli are administered to one or more locations in the left and right posterior mid-thoracic regions of a subject.

The thoracic region of the spine is the middle section of the spine, initiating at the base of the neck and extending to the base of the ribs, including a total of 12 vertebrae, identified, or labeled from T1 down to T12. The thoracic spine connects with the cervical spine above and the lumbar spine below and forms a rigid and stable structure that is attached to the rib cage. It is typically robust and less-prone to age-related mechanical damage or other non-traumatic injuries.

In exemplary forms, a mapping procedure includes identifying one or more locations in the left and right posterior mid-thoracic regions of a subject. In some forms, the anatomical location is determined by placing an electromagnetic generator/actuator device at a multiplicity of locations on the body of the subject in the mid thoracic region. In some forms, the placement of the device is in or around the T7 vertebrae.

For a typical mapping procedure, the methods include administering a single electromagnetic pulse to each of one or more testing locations on the thoracic regions of the subject and measuring a motor evoked response in the subject as an output. In some forms, the methods administer multiple pulses to each of the one or more location.

The methods typically administer electromagnetic stimuli to multiple locations within the mid thoracic regions of the subject to determine a resting motor threshold in the subject. The resting motor threshold is determined as the minimum intensity of stimulation required to achieve a motor evoked response (MEP) of a specific value in the upper rectus abdominis and external oblique muscles. Typical values of MEP that can be achieved include a voltage from between about 1 μV and about 50 μV, inclusive. In some forms, the MEP is 10 μV with 50% of trials. Typically, the resting motor threshold is determined in the upper rectus abdominis and/or external oblique muscles of the subject.

### B. Administering Electromagnetic Stimuli to Thoracic Regions

The ThorS-MagNT methods administer electromagnetic stimuli to the thoracic regions of a subject in an amount effective to increase gastrointestinal motility or to correct a neuropathy in the subject.

The methods can treat and/or prevent one or more symptoms associated with reduced gastrointestinal motility, chronic inflammatory states, autonomic neuropathy, or viscero-sensorimotor neuropathy in a subject, including refractory nausea, vomiting, early satiety, postprandial fullness, and abdominal pain. In some forms, the methods treat or prevent one or more diseases or disorders associated with reduced gastrointestinal motility, including diabetic gastroparesis, diabetic autonomic neuropathy, efferent brain-gut neuropathy and afferent gut-brain neuropathy of the subject. The methods can include administering an electromagnetic stimulus having an intensity between about 100% and about 150%, inclusive, of the minimal intensity of electromagnetic stimulation required to achieve a motor evoked response (MEP) of 10 μV in the upper rectus abdominis and/or external oblique muscles of the subject (to the extent it is possible to achieve an MEP in the subject).

The methods include administering stimuli as an electromagnetic field and other parameters typical for magnetic stimulation of a region of the thoracic cavity via stimuli applied at the skin of the subject. The methods typically include electromagnetic stimulation in an amount and intensity that does not cause damage to the body of the subject or harm the subject. In some forms, the electromagnetic stimulation is applied via an electromagnetic coil, or segment thereof. In some forms, the electromagnetic stimulation that is applied via an electromagnetic coil, or segment thereof is given by I(t)=I0·sin(2πft), with a frequency of f=10 kHz and a peak current I0=6 kA. The methods typically include an electromagnetic coil sized from 50-100 mm, such as 70 mm or 90 mm.

In some forms, the preferred and/or optimal anatomical location(s) and minimal intensity of electromagnetic stimulation required to achieve a motor evoked response (MEP) in the gastrointestinal (GI) tract of the subject are determined by mapping of the thoracic regions of the subject, as described above. Typically, the methods administer to the thoracic regions of the subject an electromagnetic stimulus having an intensity between 100% and 150% of the minimal intensity required to achieve a motor evoked response (MEP) in the rectus abdominus or external oblique muscles of the subject. If MEPs are unable to be obtained, a graded stimulus approach can be used based on the body mass index (BMI) of the subject.

In other forms, the preferred and/or optimal anatomical location and minimal intensity of electromagnetic stimulation required to achieve a motor evoked response (MEP) in the gastrointestinal (GI) tract of the subject are determined by mapping of another similar or non-similar subject. For example, in some forms, the preferred and/or optimal anatomical location and minimal intensity of electromagnetic stimulation required to achieve a motor evoked response (MEP) in the gastrointestinal (GI) tract of the subject are pre-determined and are obtained by reference to a value or database of values determined or calculated from existing studies or data. Therefore, in some forms, the methods do not require or include one or more steps for mapping the subject.

In some forms, the method includes administering between about 100 and 100,000 stimulations, or “pulses” per site to the subject via a suitable electromagnetic actuator device, such as an electromagnetic coil. The methods can include administering to one, or two, or more than two sites on the thoracic region of a subject. In some forms, the method administers between about 100 and about 100,000 stimulations, inclusive, per site to the subject. In some forms, the method administers between about 1,000 and about 20,000 stimulations, inclusive, per site to the subject. In some forms, the method administers between about 10,000 and about 15,000 stimulations, inclusive, per site to the subject. In particular forms, the method administers about 12,000 stimulations to the subject. For example, in some forms, the methods include administering 1200 pulses to two sites (i.e., to the left and right side of the thoracic region) of the subject each day for 5 days. In other forms, the methods include administering 1200 pulses to two sites per day, daily for 6 weeks. In some forms, the methods administer a total of less than 100,000 pulses per site over the entire course of treatment.

Typically, the electromagnetic stimulus includes pulses of electromagnetic stimuli having a frequency of between about 0.1 Hz and about 50 Hz, inclusive. In some forms, the electromagnetic stimulus includes pulses of electromagnetic stimuli having a frequency of between about 1 Hz and about 10 Hz, or about 20 Hz, about 30 Hz, about 40 Hz, up to 50 Hz, inclusive. For example, in some forms, the electromagnetic stimulus includes pulses of electromagnetic stimuli having a frequency of between about 0.5 Hz and about 5 Hz, inclusive. In particular forms the electromagnetic stimulus includes pulses of electromagnetic stimuli having a frequency of about 1 Hz.

In some forms, the methods obtain an MEP for the subject to determine the maximum intensity of stimuli that are applied to a subject. In other forms, e.g., when an MEP cannot be obtained, the maximum intensity of stimuli that are applied to a subject is determined on a subject-by subject basis, varied according to one or more subject-specific parameters, such as body weight, height, age, disease status, sex and body-mass index (BMI). Typically, BMI of a subject is proportionate to the maximum stimuli that are applied to the subject. Therefore, in some forms, the methods include a graded stimulus approach based on the body mass index (BMI) of the subject to determine the maximum intensity of stimuli that are applied.

In some forms, the electromagnetic stimuli are administered to the subject as a single group of consecutive pulses with the same frequency. In other forms, the electromagnetic stimuli are administered to the subject as two or more groups of stimuli, i.e., where each group of stimuli includes less than the total number of stimuli administered. Therefore, in some forms, the methods administer two or more groups of between 2 and 20,000 stimuli, where the groups are separated in time by a rest period. In some forms, each group of stimuli includes the same number of stimuli. In some forms, the groups include different numbers of stimuli. Wherein each group includes between about 10 and about 1,000 stimuli, or between about 100 and 500 stimuli. The rest period can be the same or different between consecutive groups. Exemplary rest periods include between about 10 seconds and about 30 minutes, e.g., between about 1 minute and about 10 minutes, inclusive, between about two minutes and about 7 minutes. In some forms, the two or more groups are separated in time by a rest period of about five minutes.

In an exemplary method, electromagnetic stimuli are administered at 50% intensity above motor threshold, at bilateral pre-determined locations to deliver a total of 1,200 stimulations at a frequency of about 1 Hz.

### 1. Electromagnetic Stimulation Devices

The methods typically administer electromagnetic stimuli to a subject via an approved electromagnetic actuator/stimulatory device for applying a controlled electromagnetic stimulation into the body of a subject via contact of the device with the skin or the subject in the vicinity of the anatomical location that is the target of the electromagnetic stimuli. Approved electro-magnetic actuator/stimulatory devices are known in the art and are commercially available.

In some forms, the electromagnetic actuator device is or includes an electromagnetic coil that can induce an electromagnetic field within the body of a subject by placement of the coil on the surface of the region of the body that is targeted for induction of the electromagnetic field. Multiple designs of electromagnetic coils are known in the art and can be used for described methods, including a butterfly coil, H-coil, Halo coil, and planar figure-eight coil. In some forms, the electromagnetic coil is suitable and/or marketed for transcranial magnetic stimulation (TMS). In some forms, coil is a TMS coil described in Deng, et al., Brain Stimul. 6 1-13 (2013), the content of which are hereby incorporated by reference in their entirety.

An exemplary device for mapping effective neurostimulation in the thoracic regions is the single-pulse circular 90 mm electromagnetic coil (commercially available from MAGSTIM, Whitland, UK). An exemplary device for administering electromagnetic stimuli is a 70 mm double air film self-cooling coil, such as a MAGSTIM Rapid2 device (commercially available from MAGSTIM, Whitland, UK).

In some forms, the electromagnetic coil device is held in place by a static coil fixator device throughout administration of the electromagnetic stimulation.

### C. Treatment Regimens

Systems and methods for treating one or more disease or disorders associated with reduced gastrointestinal motility, chronic inflammatory states, autonomic neuropathy, or viscero-sensorimotor neuropathy are provided. In exemplary forms, the methods improve and/or enhance gastrointestinal motility in a subject having a disease or disorder associated with chronic or acute reduced gastrointestinal motility. Exemplary diseases and disorders that can be treated include, but are not limited to, diabetic gastroparesis, diabetic autonomic neuropathy, efferent brain-gut neuropathy and afferent gut-brain neuropathy. Treatment regimens including the described methods of neuromodulation are also provided.

In some forms, the described methods of using electromagnetic stimulation for treating or preventing one or more symptoms of reduced gastrointestinal motility are administered to a subject more than once. For example, in some forms, the efficacy of the methods is determined by the number of times the treatment is administered, the timing between sequential administrations, the presence of other factors such as the administration of additional procedures and/or active agents, or combination s thereof.

Typically, treatment regimens are designed for a subject based on one or more parameters including the disease or disorder to be treated, the severity or stage of the symptoms, the intensity and amount of the electromagnetic stimuli administered and the health, age and size of the subject.

In some forms, treatment regimens include optionally mapping effective neurostimulation in the thoracic regions and administering electromagnetic stimuli to the thoracic regions one or more times. In some forms, the methods repeat only the step of administering electromagnetic stimuli to the thoracic regions one or more times. For example, in some forms, the methods include mapping effective neurostimulation in the thoracic regions once, and then administering electromagnetic stimuli to the thoracic regions of the subject multiple times

Typically, when the methods repeat one or more steps in succession, the methods include a time period between the repeating administrations that can be fixed or varied for each successive “round” of treatment. For example, in some forms, the methods include the step of administering electromagnetic stimuli to the thoracic regions, with or without a prior step of mapping effective neurostimulation in the thoracic regions, twice or more than two times in 24 hours. Therefore, in some forms, the methods are repeated once every 6 hours, 12 hours, 18 hours, 24 hours, 30 hours, 36 hours, 42 hours, or every 48 hours, for a period of one or more days, weeks, or months. The timing between rounds can be the same or different for each round. In some forms, the methods are repeated 10 times.

In an exemplary form, the methods include optionally mapping effective neurostimulation in the thoracic regions and administering electromagnetic stimuli to the thoracic regions, for example, regions identified by mapping, every 12 hours for a period of five consecutive days. In some forms, the methods include administering electromagnetic stimuli to the thoracic regions identified in the mapping every 12 hours for a period of five consecutive days. In some forms, the methods include the step of administering electromagnetic stimuli to the thoracic regions without performing a prior mapping step. In some forms, the methods repeat the step of administering electromagnetic stimuli to the left and right posterior mid-thoracic regions of a subject a subject every 12 hours for a period of five consecutive days. Typically, the administration includes placing an electromagnetic actuator device, such as a coil, onto the skin of the left and right posterior mid-thoracic regions of a subject. In some forms, when the methods do not include mapping effective neurostimulation in the thoracic regions, this information is obtained based on prior information relevant to the subject or to a similar or correlative subject. In some forms the treatment regimen includes a total number of administrations of electromagnetic stimuli given to a subject over a period of time, e.g., 20 administrations over a period of 10 days, or 10 administrations over 5 days, or 5 administrations over 5 days. In some forms, the same or different treatment, i.e., including the same or different number of stimuli, each having the same or different intensity, is administered to a subject at the same or different time every day for a period of two or more days, such as three, four, five, six, seven, eight, nine, or ten consecutive days, or more than ten consecutive days, such as 11, 12, 13, 14, 15, 16, 17, 18, 19, 20, or 21 days, or for up to six weeks. In some forms, the same treatment, i.e., including the same number of stimuli, each having the same intensity, is administered to a subject at the same or different time every day for a period of two or more days, such as three, four, five, six, seven, eight, nine, or ten consecutive days, or more than ten consecutive days, such as 11, 12, 13, 14, 15, 16, 17, 18, 19, 20, or 21 days, or for up to six weeks.

In an exemplary method, electromagnetic stimuli are administered at 50% intensity above motor threshold, at bilateral pre-determined locations to deliver a total of 1,200 stimulations at a frequency of about 1 Hz in groups of 300 stimuli separated by a rest period of 5 minutes, and the method is carried out twice or more times a day for 5 days.

In some forms, the method optionally includes one or more steps for obtaining and recording one or more pieces of data relating to the progress and/or outcome of the methods. For example, in some forms the methods record safety, efficacy and/or tolerability data from the subject. Safety, efficacy, and tolerability data obtained by a treating physician or recorded directly by the subject are useful to optimize and improve the therapeutic efficacy of the methods as administered to each subject. In some forms, the methods include one or more steps for assessing the efficacy of treatment as determined by the amelioration of one or more symptoms, diseases or disorders in the subject.

### D. Diseases and Disorders to be Treated

The methods administer electromagnetic stimuli to the thoracic regions of a subject in an amount effective to increase gastrointestinal motility and to improve neuropathy in the subject. Therefore, methods to treat and prevent one or more diseases and disorders associated with reduced or impaired gastrointestinal motility are provided.

In some forms, the methods treat or prevent one or more of diabetic autonomic neuropathy, efferent brain-gut neuropathy and/or afferent gut-brain neuropathy in a subject in need thereof. In some forms, the methods treat or prevent one or more symptoms selected from sphincter dysfunction, nausea, vomiting, retching, early satiety, postprandial fullness, bloating, loss of appetite, weight loss, abdominal distension and upper abdominal pain. In a preferred form, the methods reduce or prevent one or more of refractory nausea, vomiting, early satiety, postprandial fullness, and abdominal pain in a subject.

The symptoms can be chronic or acute. Therefore, in some forms, the methods are effective to reduce the severity of one or more acute or chronic symptoms and/or episodes of symptomatic events in a subject over a period of time. In some forms the methods reduce the number of vomiting episodes (emesis) in the subject over a period of 12 hours, 24 hours, 48 hours or 72 hours.

In some forms, the methods are effective to provide a therapeutic benefit to a subject for a period of time. For example, in some forms, the methods treat or prevent the one or more symptoms in the subject for a time selected from one day, three days, five days, one week, two weeks, three weeks, four weeks, one month, two months, three months, four months, five months, six months, or one year, or more than one year. In some forms, the methods treat or prevent one or more symptoms associated with reduced or impaired gastrointestinal motility for up to three weeks, up to six weeks, or up to six months. In some forms, the methods reduce one or more symptoms in a subject for a period of time following administration of the methods, for example, foe one or more days, weeks, months or years following treatment. In some forms, reduction or prevention of one or more symptom in a subject is achieved for a period of 1, 2, 3, 4, 5, 6, 7, 8, 9, 10, or more than 10 days, weeks, or years following administering the methods to the subject. In some forms, the subject is treated by repeating administration of the methods to the subject, for example, by repeating the methods once or more every 1, 2, 3, 4, 5, 6, 7, 8, 9, 10 or more days, weeks, months or years. In an exemplary form, a subject is treated once every year, or every two years, or every three years or every four years.

### 1. Subjects in Need of Treatment

Any of the methods described herein can include one or more steps of selecting a subject in need of treatment. In some forms, a subject in need of treatment is a patient having one or more symptoms associated with reduced or impaired gastrointestinal motility chronic inflammatory states, autonomic neuropathy, or viscero-sensorimotor neuropathy. Typically, the subject is a human, such as a human adult or a human child. The results and/or efficacy of the methods of treatment can be compared to a control, such as an untreated subject having reduced or impaired gastrointestinal motility. In some forms, the results and/or efficacy of the methods of treatment are compared to a healthy control subject who does not and has not had reduced or impaired gastrointestinal motility. In other forms, the results and/or efficacy of the methods of treatment are compared to a subject who has had reduced or impaired gastrointestinal motility and has recovered. Typically, the subject does not have one or more ulcers in the stomach or elsewhere in the GI tract.

Typically, the methods are administered to a subject who does not have one or more physical obstructions and/or constrictions in the stomach or elsewhere in the GI tract, or severe cardiac disease and arrhythmias, or a metal implant or prosthesis.

In some forms, the subject has been diagnosed as having, or as being at increased risk of having, or is suspected as having a disease or disorder associated with reduced gastrointestinal motility. In some forms, the subject has been diagnosed as having, or as being at increased risk of having, or is suspected as having irritable bowel syndrome (IBS). In some forms, the subject has been diagnosed as having, or as being at increased risk of having, or is suspected as having gastroparesis, such as diabetic gastroparesis. In some forms, the subject has been diagnosed as having, or as being at increased risk of having, or is suspected as having a visceral neuropathy and/or myopathy. In some forms, the subject has one or more symptoms of nausea, retching, vomiting, stomach fullness, inability to finish a meal, excessive fullness, loss of appetite, bloating and abdominal distension, abdominal pain, and early satiety. The symptoms can be acute or chronic. In some forms, the subject is asymptomatic. For example, in some forms, the patient has a history of symptoms associated with reduced gastrointestinal motility but has no symptoms at the point of treatment. Therefore, in some forms the methods prevent or delay the onset of one or more of the symptoms associated with reduced gastrointestinal motility. For example, in some forms, the subject has received surgery, and/or is taking medications that slow gastric emptying and cause similar symptoms, such as opioid pain relievers, antidepressants, high blood pressure medication, and/or allergy medications. In some forms, the methods to select a subject include one or more steps of administering a barium meal, or an egg meal to the subject to identify a delay in meal passing.

### 2. Gastroparesis

In some forms, the methods reduce or prevent the occurrence or persistence of a symptom in a subject having gastroparesis.

Gastroparesis is a symptomatic condition of delayed gastric emptying with no mechanical obstruction (Parkman H, et al. Gastroenterology 2004;127:1592-1622; Camilleri M, et al., Am J Gastroenterol 2013;108:18-37). There are several etiologies of gastroparesis, including diabetic gastroparesis and postsurgical gastroparesis. In many patients, a cause cannot be found, and the condition is termed idiopathic gastroparesis. In some of these patients, a viral etiology may be suspected due to a sudden onset of symptoms associated with a viral-like prodrome. A variety of symptoms are reported by patients with gastroparesis (Parkman, et al. Clin Gastroenterol Hepatol. 2011 December;9(12):1056-64). These can include nausea, vomiting, early satiety, postprandial fullness, bloating, loss of appetite, abdominal distension, and abdominal pain. Patients may experience any combination of symptoms with varying degrees of severity. The symptoms are often chronic; however, patients may also have periodic exacerbations of their symptoms. These symptoms reduce the patient's health-related quality of life. Many patients experience weight loss due to their symptoms. Some of the possible complications of gastroparesis can be life-threatening, such as malnutrition, dehydration, electrolyte imbalances and blood sugar fluctuations with diabetes.

In some forms, gastroparesis in the subject is associated with, or is a result of a condition, disease or disorder, injury or surgery in the subject. Any of the methods described herein can include one or more methods of identifying a subject for treatment. Methods for identifying and/or diagnosing gastroparesis in a subject are known in the art, and in some forms, the subject is diagnosed as having, or being at increased risk of having gastroparesis. Typically, a diagnosis of gastroparesis is made by demonstrating in a symptomatic patient delayed gastric emptying without evidence of obstruction. Delayed gastric emptying is most commonly assessed using a validated measurement of gastric emptying. At present, the best validated, and approved method of measurement is scintigraphy of the solid phase of a meal. Other ways to assess gastric emptying include wireless capsule motility and breath tests using stable C-13 isotopes. Absence of obstruction is most commonly determined by upper endoscopy; an alternative test is an upper gastrointestinal radiographic series, which can also assess the small intestine.

In some forms, the subject has an underlying medical condition, including diabetes, HIV/AIDS, hypertension, viral infection, bacterial infection, fungal infection, or another chronic metabolic or immune disorder. In some forms, the subject has diabetes. In some forms, the subject has gastroparesis associated with a trauma, injury or surgery. In some forms, the subject has idiopathic gastroparesis. In some forms, the subject has diabetic gastroparesis. In some forms, the subject does not have severe cardiac disease and arrhythmias. In some forms, the subject does not have a metal implant or prosthesis.

### 3. American Neuro-gastroenterology and Motility Society Gastroparesis Cardinal Symptom Index Daily Diary (ANMS GCSI-DD)

In some forms the methods are effective to treat or prevent one or more symptoms identified in the American Neuro-gastroenterology and Motility Society (ANMS) Gastroparesis Cardinal Symptom Index Daily Diary (ANMS GCSI-DD). In gastroparesis, the symptom experience and severity can be obtained from the patient. Therefore, patient-reported symptom scales that capture overall gastroparesis severity are helpful for evaluating treatments for gastroparesis. Therefore, in some forms, the methods include one or more steps of monitoring and/or recording occurrence or changes in one or more symptoms in a patient. The monitoring and/or recording can include use of the ANMS GCSI-DD.

The Gastroparesis Cardinal Symptom Index (GCSI) was developed to assess the core symptoms of gastroparesis and represents a subset of the longer, 20-item, Patient Assessment of Upper Gastrointestinal Disorders Symptoms (PAGI-SYM) questionnaire, which was developed to assess symptoms of gastroparesis, functional dyspepsia and gastroesophageal reflux disease. The GCSI quantifies the severity of nine gastroparesis symptoms: nausea, retching, vomiting, stomach fullness, inability to finish a meal, excessive fullness, loss of appetite, bloating and abdominal distension. Therefore, in some forms, the methods treat or prevent one or more of nausea, retching, vomiting, stomach fullness, inability to finish a meal, excessive fullness, loss of appetite, bloating and abdominal distension in a subject in need thereof. In some forms, the methods include one or more steps to measure ANMS GCSI-DD at baseline and throughout a treatment regimen, and/or monitoring and/or recording the progression, emergence, or other changes in symptoms.

Therefore, in some forms, the method is effective to improve one or more symptoms identified in the ANMS GCSI-DD by one or more points. For example, when ANMS GCSI-DD points are defined as 0=none, 1=mild, 2=moderate, 3=severe, and 4=very severe, the methods are effective to improve one or more of the symptoms identified in the ANMS GCSI-DD by reduction of one point, two points, three points, or four points. In some forms, the baseline ANMS GCSI-DD is determined before treatment, and the ANMS GCSI-DD is measured during and/or after treatment. In some forms, the methods reduce the ANMS GCSI-DD relative to baseline by from about 1% to about 100%, inclusive. For example, in some forms, the methods reduce the ANMS GCSI-DD relative to baseline by 30% or more than 30%, such as 40%, 50%, 60%, 70%, 80%, 90% or 95%.

In some forms, the methods are effective to treat or prevent the one or more symptoms in the subject identified in the GCSI for a time selected from one day, three days, five days, one week, two weeks, three weeks, four weeks, one month, two months, three months, four months, five months, six months, or one year, or more than one year, such as two years, three years, or four years. In some forms, the methods treat or prevent multiple symptoms associated with gastroparesis for up to three weeks, up to six weeks, or up to six months or one year, or more than one year, such as up to two years, up to three years, or up to four years.

### 4. Other Visceral Neuropathies or Myopathies

In some forms, the methods administer electromagnetic stimuli to the thoracic region to effectively treat one or more symptoms of one or more visceral neuropathies in a patient in need thereof.

Visceral neuropathies include diseases of the nerves affecting mostly the internal organs such as the bladder muscles, the cardiovascular system, the digestive tract, liver, pancreas and the genital organs. These nerves are not under a person's conscious control and function automatically. Visceral neuropathies may involve the smooth muscles of the whole or specific sections of the GI tract and occasionally the smooth muscles of the urinary tract. In some forms, the nerves affected control blood pressure, heart rate, temperature, in addition to digestion. Therefore, in some forms, the subject has symptoms including pain as well as abnormalities in sweat production, impotence, abnormal vision, abnormal heart rate, abnormal blood pressure, and abnormal respiration.

Visceral myopathies are diseases where severe visceral smooth muscle dysfunction prevents efficient movement of air and nutrients through the bowel, impairs bladder emptying, and affects normal uterine contraction and relaxation, particularly during pregnancy. Disease severity exists along a spectrum and visceral myopathy may mimic other GI disorders. In some forms, a visceral myopathy is present with long-lasting or recurrent episodes of abdominal distension without evidence of mechanical obstruction, constipation and intermittent abdominal pain.

Therefore, in some forms, the methods treat or prevent one or more symptoms coupled with nausea, vomiting, abdominal pain, early satiety, postprandial fullness, bloating and distention in a patient diagnosed as having, or suspected as having a visceral neuropathy or myopathy. In some forms, the subject has one or more disease or disorder including, but not limited to, irritable bowel syndrome (IBS), functional dyspepsia, idiopathic gastroparesis, postsurgical gastroparesis, non-alcoholic fatty liver disease (NAFLD), non-alcoholic steatohepatitis (NASH), chronic hepatitis, chronic pancreatitis, chronic idiopathic constipation, colonic inertia, chronic intestinal pseudo-obstruction, cardiac autonomic neuropathy, adrenal insufficiency, interstitial cystitis, achalasia, esophageal dysmotility, noncardiac chest pain, gastroesophageal reflux disease, anorexia nervosa, bulimia, avoidant restrictive food intake disorder, inflammatory bowel disease, and disorders of gut-brain interaction (DGBI).

### E. Additional Procedures and Active Agents

Any of the described methods can include the administration of one or more additional therapeutic procedures and/or active agents to the subject.

In some forms, the methods include one or more procedures for the management of reduced or impaired gastric contractility, autonomic neuropathy, efferent brain-gut neuropathy, afferent gut-brain neuropathy, gastroparesis or one or more symptoms thereof in the subject.

In some forms, the methods include, or are administered together with one or more procedures including changes to diet (e.g., removal of certain foods and/or increased consumption of foods that are easier to digest; eating smaller meals more frequently; chewing food thoroughly; eating well-cooked fruits and vegetables rather than raw fruits and vegetables; consumption of low-fat foods); exercise after eating, avoid lying down for two hours after a meal; mental and physical relaxation techniques, such as mindful meditation and/or yoga, and daily consumption of a multivitamin.

In some forms, the methods include administering one or drugs to the subject. The administration of the drug can be prior to, during or after the described methods for electromagnetic neurostimulation. Exemplary active agents that can be administered to the subject in addition to the described methods include agents to stimulate the stomach muscles (such as metoclopramide (REGLAN) erythromycin, and domperidone); agents to control nausea and vomiting (such as diphenhydramine (BENADRYL), ondansetron (ZOFRAN), and Prochlorperazine (COMPRO)); anti-microbial agents, anesthetic agents, neuromodulator agents (antidepressants, antipsychotics, anti-anxiolytics, anti-epileptic agents, and other agents for neuropathic pain), or combinations thereof. In certain forms, the additional active agent is Metoclopramide.

The methods will be better understood in view of the following paragraphs:

1. A method for treatment of gastroparesis in a subject including electrical stimulation of the thoracic sympathetic and/or parasympathetic nerves of the subject.
2. The method of paragraph 1, including
(i) mapping of the thoracic regions of the subject, wherein the mapping includes identifying a preferred and/or optimal anatomical location(s) and minimal intensity of electrical stimulation required to achieve a motor evoked response (MEP) value in the gastrointestinal (GI) tract of the subject; and
(ii) administering to the location identified in (i) an electrical stimulus having an intensity between 100% and 150% of the minimal intensity identified in (i).
   3. The method of paragraph 2, wherein the optimal anatomical location(s) within the thoracic regions of the subject includes the left and right posterior mid-thoracic regions.
   4. The method of paragraph 2 or 3, wherein the MEP value is between about 1 μV and about 50 μV, inclusive.
   5. The method of any one of paragraphs 2-4, wherein the MEP value is about 10 μV.
   6. The method of any one of paragraphs 2-5, wherein the MEP is evoked in the upper rectus abdominis and/or external oblique muscles of the subject.
   7. The method of any one of paragraphs 2-6, wherein the electrical stimulation in (i) includes electrical stimulation applied to the subject through an electromagnetic coil.
   8. The method of paragraph 7, wherein the electromagnetic coil includes a single-pulse circular 90 mm electromagnetic coil.
   9. The method of any one of paragraphs 2-8, wherein the electrical stimulation in (ii) includes electrical stimulation applied to the subject through an electromagnetic coil.
   10. The method of paragraph 9, wherein the electromagnetic coil includes a 70 mm double air film self-cooling electromagnetic coil.
   11. The method of any one of paragraphs 7-10, wherein the electromagnetic coil is held in place by a static coil fixator device throughout administration of the electrical stimulation.
   12. The method of any one of paragraphs 2-11, wherein the electrical stimulus in step (ii) includes between about 100 and 100,000 stimulations, inclusive, or between 100 and about 100,000 stimulations, inclusive, or between about 1,000 and about 20,000 stimulations, inclusive.
   13. The method of paragraph 12, wherein the electrical stimulus in step (ii) includes between about 10,000 and about 15,000 stimulations, inclusive.
   14. The method of paragraph 12 or 13, wherein the electrical stimulus in step (ii) includes about 12,000 stimulations.
   15. The method of any one of paragraphs 2-14, wherein the electrical stimulus in step (ii) has a frequency of between about 0.1 Hz and about 10 Hz, inclusive, or between about 0.5 Hz and about 5 Hz, inclusive.
   16. The method of any one of paragraphs 2-15, wherein the electrical stimulus in step (ii) has a frequency of about 1 Hz.
   17. The method of any one of paragraphs 2-16, wherein the electrical stimulus in step (ii) is administered to the subject in two or more groups of pulses,
wherein each group includes less than the total number of pulses, and
wherein the two or more groups are separated in time by a rest period.
   18. The method of paragraph 17, wherein each group includes between about 10 and about 1,000 pulses, or between about 100 and 500 pulses.
   19. The method of paragraph 17 or 18, wherein each group includes about 300 pulses.
   20. The method of any one of paragraphs 17 to 19, wherein the rest period is between about 10 seconds and about 30 minutes, inclusive, or between about one minute and about ten minutes, inclusive.
   21. The method of paragraph 20, wherein the rest period is about five minutes.
   22. The method of any one of paragraphs 2-21, further including repeating the electrical stimulation of the thoracic sympathetic and/or parasympathetic nerves of the subject once or more than once.
   23. The method of paragraph 22, wherein repeating the electrical stimulation includes both
(i) mapping of the thoracic regions of the subject; and
(ii) administering to the location identified in (i) an electrical stimulus having an intensity between 100% and 150% of the minimal intensity identified in (i).
   24. The method of paragraph 22, wherein repeating the electrical stimulation does not include
   mapping of the thoracic regions of the subject.
   25. The method of any one of paragraphs 2-24, wherein the electrical stimulation of the thoracic sympathetic and/or parasympathetic nerves of the subject is repeated at a regular interval.
   26. The method of paragraph 25, wherein the interval is selected from the group including one hour, 12 hours, one day, two days, one week, two weeks, three weeks, one month, two months, six months and one year.
   27. The method of paragraph 22, wherein the methods administer steps (i) and (ii) to a subject two times a day for a period of five consecutive days.
   28. The method of any one of paragraphs 2-27, further including administering to the subject one or more additional therapeutic agents or procedures.
   29. The method of paragraph 28, wherein the additional therapeutic agent and/or procedure is administered to the subject at the same time, before or after the electrical stimulation of the thoracic sympathetic and/or parasympathetic nerves.
   30. The method of paragraph 29, including administering one or more additional therapeutic agents or procedures to the subject during the electrical stimulation of the thoracic sympathetic and/or parasympathetic nerves.
   31. The method of any one of paragraphs 28-30, wherein the additional active agent is selected from the group including a prokinetic agent, an antiemetic agent, an anti-microbial agent, ana an anesthetic agent or combinations thereof.
   32. The method of any one of paragraphs 28-31, wherein the additional active agent is Metoclopramide.
   33. The method of any one of paragraphs 1-32, wherein the method is effective to treat or prevent one or more symptoms of gastroparesis selected from the group including anal sphincter dysfunction, nausea, vomiting, retching, early satiety, postprandial fullness, bloating, loss of appetite, weight loss, abdominal distension and upper abdominal pain.
   34. The method of paragraph 33, wherein the method is effective to reduce the number of vomiting episodes (emesis) in the subject over a period of time selected from the group including 12 hours, 24 hours, 48 hours and 72 hours.
   35. The method of paragraph 33, wherein the method is effective to treat or prevent one or more symptoms identified in the ANMS Gastroparesis Cardinal Symptom Index (ANMS GCSI-DD).
   36. The method of paragraph 35, wherein the method is effective to improve one or more of the symptoms identified in the ANMS GCSI-DD by one or more points,
wherein the points are defined as 0=none, 1=mild, 2=moderate, 3=severe, and 4=very severe.
   37. The method of paragraph 36, wherein the method is effective to improve one or more of the symptoms identified in the ANMS GCSI-DD by reduction of four points.
   38. The method of any one of paragraphs 33-37, wherein the method is effective to treat or prevent the one or more symptoms in the subject for a time selected from the groups including one day, three days, five days, one week, two weeks, three weeks, four weeks, one month, two months, three months, four months, five months, six months, and one year.
   39. The method of any one of paragraphs 33-38, wherein the subject is a symptomatic patient having delayed gastric emptying without evidence of obstruction, as determined by a gastric emptying breath test (GEBT), upper endoscopy, or an upper gastrointestinal radiographic series.
   40. The method of any one of paragraphs 33-39, wherein the subject has been diagnosed as having, or is suspected as having idiopathic gastroparesis, diabetic gastroparesis, or surgical gastroparesis.
   41. The method of any one of paragraphs 33-40, wherein the subject has been diagnosed as having diabetic gastroparesis.
   42. The method of any one of paragraphs 33-41, wherein the subject does not have irritable bowel syndrome (IBS), severe cardiac disease and arrhythmias, and/or
wherein the subject does not have a metal implant or prosthesis.
   43. A method to treat and prevent efferent spinogastric neuropathy and afferent gastro-cortical neuropathy in a subject including administering to the upper rectus abdominis and/or external oblique muscles of the subject electrical stimuli having an intensity between 100% and 150% of the minimal intensity of electrical stimulation required to achieve a motor evoked response (MEP) of 10 μV in the upper rectus abdominis and/or external oblique muscles of the subject.
   44. The method of paragraph 43, wherein the electrical stimuli include about 1,200 pulses having a frequency of 1 Hz.
   45. The method of paragraph 44, wherein the stimuli is delivered in groups of 300 pulses separated by a rest period of 5 minutes.
   46. The method of paragraph 45, wherein the subject has been diagnosed as having diabetic gastroparesis.

The description will be better understood in view of the following working examples.

### EXAMPLES

### Example 1: Treatment Using Repetitive Magnetic Stimulation for Diabetic Gastroparesis Achieved Reduction in ANMS GCSI-DD Score

### Methods

DGp patients, who required hospital admission and without known GI mucosal disease, were included. A mapping procedure in the left and right posterior mid-thoracic regions, using a single-pulse circular 90 mm coil (MAGSTIM, Whitland, UK) determined the location and minimum intensity required to achieve a motor evoked response (MEP) of 10 mV with 50% of trials (resting motor threshold) in the upper rectus abdominis and external oblique muscles. ThorS-MagNT was administered at 50% intensity above motor threshold, using a 70 mm double air film self-cooling coil (MAGSTIM Rapid2) positioned over bilateral pre-determined locations to deliver a total of 1,200 stimulations at 1 Hz. Treatments were administered twice daily for five consecutive days. Safety and tolerability data were collected. ANMS GCSI-DD was assessed at baseline and throughout the treatment period. Patients who completed all ten treatments were defined as per protocol.

### Results

Seven DGp patients (6 Female; 1 Male) were enrolled. All subjects tolerated administered treatment sessions. One DGp patient experienced medication-related hypotension-a serious adverse event unrelated to study treatment. After resuscitation and missing one treatment, this subject completed 4 subsequent treatments. One patient reported self-limited tingling and numbness, which resolved after treatment. No other adverse events occurred. In four per protocol subjects, an average of 68.25% reduction in ANMS GCSI-DD score from baseline was observed (Table 1). Two other subjects had an average of 13.95% reduction in ANMS GCSI-DD and one study subject dropped out.

### Conclusion

Based on this study, ThorS-MagNT appears to be a feasible, safe, promising new treatment for refractory DGp. See Table 1.

TABLE 1
ANMS GCSI-DD scores at baseline versus during treatment

| | | During | | |
|---|---|---|---|---|
| | Baseline | Treatment | Δ¹ | |
| Subject | (B) | (DT) | (B − DT) | Δ¹% |
| AUTN0001 | — | 3.2 | — | — |
| AUTN0002 | 3.32 | 2.64 | 0.68 | 20.5 |
| AUTN0003 | 3.24 | 3.0 | 0.24 | 7.4 |
| AUTN0004 | 3.12 | 0.76 | 2.36 | 75.6 |
| AUTN0005 | 1.3 | 0.3 | 1.0 | 76.9 |
| AUTN0006 | 1.2 | 0.4 | 0.8 | 66.6 |
| AUNTN007 | 2.6 | 1.2 | 1.4 | 53.8 |

### Example 2: Thoracic Spinal Nerve Neuromodulation Therapy is Effective at Treating Diabetic Gastroparesis

Translumbosacral neuromodulation therapy (TNT) corrects underlying bidirectional gut-brain neuropathy (efferent spinal-anorectal neuropathy and afferent ano-cortical neuropathy) and anal sphincter dysfunction by treatment of lumbosacral spinal nerve roots to improve fecal incontinence. Experiments were designed to determine if targeting bilateral nerve roots at the mid-thoracic level with repetitive magnetic stimulation would reverse efferent spinal-gastric neuropathy and afferent gastro-cortical neuropathy to improve symptoms of gastroparesis. Thoracic Spinal Nerve Magnetic Neuromodulation Therapy (ThorS-MagNT) is a new neuromodulation treatment, which uses a magnetic coil to deliver repetitive stimulations to spinal nerves in the mid-thoracic, paraspinal regions of the back in a similar fashion to transcranial magnetic stimulation (TMS) delivering repetitive magnetic stimulation to various regions of the skull. TMS is supported by strong evidence for the treatment of depression and neuropathic pain (Lefaucheur, et al., Clinical Neurophysiology 2014;125:2150-2206).

### Methods

Refractory diabetic gastroparesis (DGp) subjects were recruited into the study approved by the Augusta University Institutional Review Board (IRB No. 1623939) and registered in Clinicaltrials.gov (NCT04706832). Inclusion criteria were previously diagnosed DGp in subjects less than 85 years with refractory symptoms despite treatment or intolerance to treatment. Exclusion criteria were postsurgical or idiopathic gastroparesis, gastrointestinal obstruction or presence of gastric bezoar, active inflammatory bowel disease or other mucosal disease, use of opioids, active depression, severe cardiac disease and arrhythmias, presence of metal implants (gastric electrical stimulators, deep brain stimulators, sacral nerve stimulators, or cardiac pacemakers), and pregnant women or nursing mothers. If dosage was stable for greater than 3 months, patients were permitted to continue medications affecting gastric emptying. If subjects met inclusion criteria, they signed an informed consent and were enrolled into the study. Subjects kept a 7-day baseline ANMS Gastroparesis Cardinal Symptom Index Daily Diary (ANMS GCSI-DD) prior to treatment and for an additional 21 days, which was completed every evening 1 hour after dinner. The ANMS GCSI-DD is a validated patient-reported outcome measure of cardinal symptoms of gastroparesis, including nausea, early satiety, post-prandial fullness and upper abdominal pain (0-4, increasing severity) and number of vomiting episodes (0-4, with greater than 4 episodes recorded as 4) (Revicki, et al., Neurogastroenterology & Motility 2019;31:e13553). The FDA-responder definition for gastroparesis clinical trials is a 30% or greater reduction in 7-day total ANMS GCSI-DD score. Subjects also completed a device tolerability questionnaire during treatment.

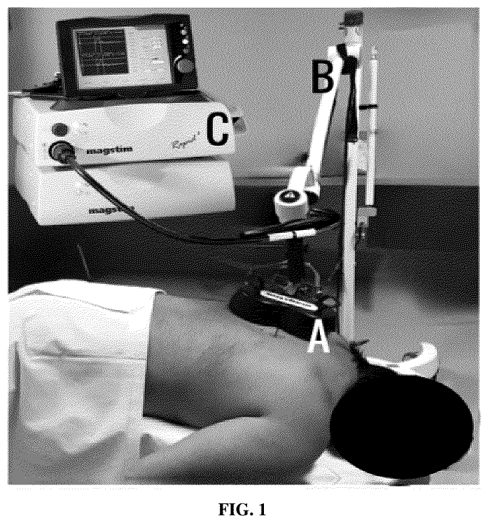

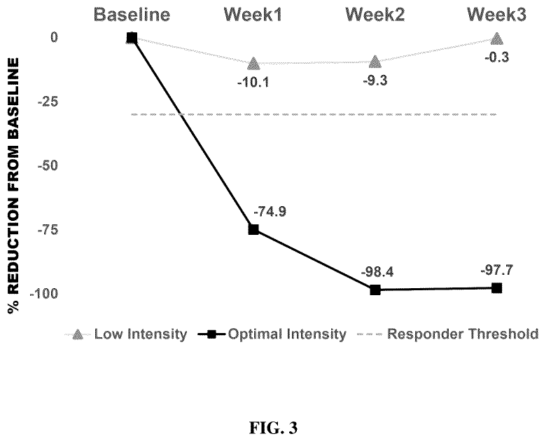

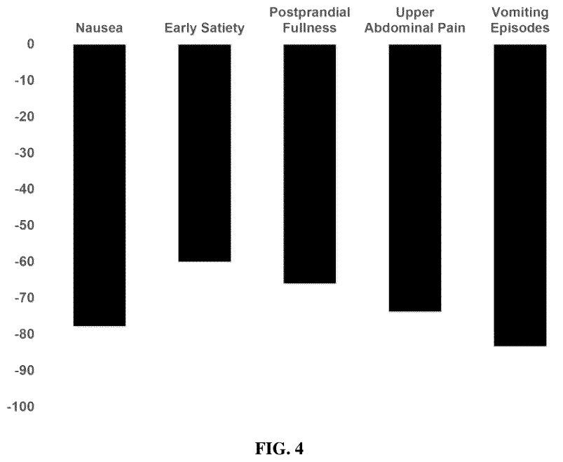

ThorS-MagNT treatment was administered twice daily for five consecutive days, following evidence-based transcranial magnetic stimulation (TMS) treatment schedules for depression and neuropathic pain (Lefaucheur et al., Clinical Neurophysiology 2014;125:2150-2206). The inferior angles of the scapula served as landmarks for the T7 level. The resting motor thresholds were obtained using a single-pulse, circular, 90-mm, stimulation coil (Magstim 200; MAGSTIM, Whitland, UK) to determine the minimum intensity of stimulation required to achieve a motor response of 10 μV with 50% of trials in the upper rectus abdominis and external oblique muscles. If obtained, the maximum intensity used for ThorS-MagNT treatment did not exceed 150% above this motor threshold, in accordance with repetitive magnetic stimulation safety guidelines. Thoracic stimulations were administered with a 70-mm double air film self-cooling coil (MAGSTIM Rapid2, Whitland, UK) positioned on marked bilateral locations, held in place by a coil fixator to deliver, as shown in FIG. 1. Stimulation settings were two trains of 300 pulses at 1 Hz frequency with an intermittent 5-min rest period (Total dose 1,200 stimulations per session) for 10 total sessions over 5 days. Treatment frequency was derived from prior repetitive magnetic treatment experience of the lumbosacral region in a dose-ranging study for fecal incontinence, demonstrating that the 1 Hz was the most efficacious frequency setting (Rao, et al., Official journal of the American College of Gastroenterology! ACG 2021;116:162-170). Patient were continuously monitored via cardiorespiratory monitoring throughout the study and closely observed for any adverse events. Gastric emptying was assessed through gastric emptying breath tests one week prior to and 2 weeks after treatment, as shown in FIGS. 3-4.

### Results

Seven DGp patients (6 Female, 1 Male) with moderate-severe gastroparesis (average baseline total ANMS GCSI-DD≥2.0) were enrolled. Patient demographics, pertinent past medical history details, medication histories, and response to treatment are shown in Table 2. All subjects completed treatment and tolerated treatment sessions. All subjects (n=4) treated at optimal intensities of 150% above motor threshold met FDA-responder definition of achieving greater than 30% reduction in total ANMS GCSI-DD. These responders had an average of 74.9% reduction in total ANMS GCSI-DD score from baseline (FIG. 3) versus 10.1% reduction in subjects treated with low intensities (at threshold level). None of the subjects had worsening of symptoms. Symptom improvement persisted 2 weeks post-treatment with an average of 97.9% symptom reduction in responders (FIG. 3) versus 0.3% reduction in subjects treated with low intensities. ThorS-MagNT responders had greater than 60.0% improvement in all four gastroparesis cardinal symptom domains (nausea, early satiety, postprandial fullness, and upper abdominal pain) and 83.3% improvement in number of vomiting episodes (FIG. 4). The most improved symptom was nausea (77.8%), as seen with prior gastric electrical stimulation studies (Levinthal, et al., Autonomic Neuroscience 2017;202:45-55). Upper abdominal pain, a refractory gastroparesis symptom, also significantly improved by 73.8% from baseline. Three of four responders had a 60.4% improvement in t1/2 on gastric emptying breath test (GEBT). One subject with known hypertension experienced medication-related hypotension, unrelated to study treatment. After resuscitation, this subject completed four remaining treatments. Another subject reported self-limited tingling and numbness, which resolved after treatment. No other adverse events occurred. All seven subjects tolerated the treatment well and marked ‘agree’ with statement ‘ThorS-MagNT treatment was easy and convenient’.

In this study, a technique of delivering a promising, non-invasive, safe, and feasible neuromodulatory treatment, Thoracic Spinal nerve Magnetic Neuromodulation Therapy (ThorS-MagNT) was applied to subjects with refractory DGp. Subjects treated with optimal treatment intensity demonstrate an excellent therapeutic response with a significant and sustained improvement in gastroparesis cardinal symptoms and report excellent tolerance. There is a high placebo response in gastroparesis and other disorders of gut-brain interaction (DGBI) (Kaptchuk, et al., Bmj 2008;336:999-1003). Symptom response in the study was durable, persisting greater than 21 days post-treatment. The clinical observation is that ThorS-MagNT responders maintain symptom relief for greater than 6 months. This durable response to treatment indicates that a placebo effect less likely. Nonetheless, a sham comparison is desired. Limitations of the study include its unblinded nature and small sample size. This proof-of-concept study of ThorS-MagNT in subjects with diabetic gastroparesis offers promise of a non-invasive, safe, efficacious, and durable treatment for a debilitating condition. The anticipated results of a larger, multi-site, randomized, sham-controlled trial, the TNM-DGp (Thoracic Neuromodulation for Diabetic Gastroparesis) trial, Clinicaltrials.gov NCT05273788, will further shed light on the safety, feasibility, and efficacy and allow better understanding of the mechanism of action of thoracic neuromodulation.

TABLE 2
Patient demographics, pertinent past medical history details, medication histories, responder status, and
magnitude of response defined as percent symptom (total ANMS GCSI-DD score) improvement from baseline.

| | | | | | | | |
|---|---|---|---|---|---|---|---|
| Age | 24 | 48 | 57 | 48 | 25 | 56 | 60 |
| Gender | F | F | F | F | F | F | M |
| Type of | Type 1 | Type 2 | Type 2 | Type 2 | Type 1 | Type 2 | Type 2 |
| Diabetes | | | | | | | |
| Duration of | 3 years | 2 years | 6 years | 8 years | 3 years | 3 years | 1 years |
| diabetic | | | | | | | |
| gastroparesis | | | | | | | |
| HbA1c | 9 | 7.4 | 6.4 | 5.6 | 9.5 | 6.1 | 6.8 |
| Co-morbidities | Adjustment | Anxiety | CAD | Gout | Hypertension | Bipolar | Hypertension |
| | disorder | Hyperlipidemia | Migraines | Depression | | 1 disorder | |
| | Endometriosis | Hypertension | Hypertriglyceridemia | Hypothyroidism | | | |
| | | | Obstructive | Osteochondritis | | | |
| | | | sleep apnea | dissecans | | | |
| | | | Adrenocortical | | | | |
| | | | insufficiency | | | | |
| | | | Hypothyroidism | | | | |
| Medications | Carafate | Albuterol | Aspirin | Allopurinol | Insulin | Atorvastatin | Amlodipine |
| (Stable dosage >3 | Carvedilol | Budesonide- | Atorvastatin | Atorvastatin | Pantoprazole | Cyclobenzaprine | Carvedilol |
| months) | Ferrous sulfate | formoterol inh | Brexpiprazole | Buspirone | | Dexlansoprazole | Gabapentin |
| | Gabapentin | Buprenorphine | Carvedilol | Colchicine | | Dulaglutide | Glipizide |
| | Hydroxyzine | Citalopram | Cetirizine | Dapagliflozin | | Duloxetine | Insulin |
| | Insulin | Estradiol | Cholecalciferol | Dextroamphetamine | | Empagliflozin | Lubiprostone |
| | Losartan | Insulin | Cholestyramine | Duloxetine | | Gabapentin | Nortriptyline |
| | Pantoprazole | Nortriptyline | Erenumab-aooe inj | Famotidine | | Glimepiride | Pantoprazole |
| | Promethazine | Pantoprazole | Fluticasone nasal | Flonase | | LamotriIgine | Promethazine |
| | Prucalopride | Phenazopyridine | Furosemide | Gabapentin | | Linaclotide | Tadalafil |
| | | Pregabalin | Isosorbide mononitrate | Insulin | | Lurasidone | |
| | | Promethazine | Levomilnacipran | Levothyroxine | | Promethazine | |
| | | Simvastatin | Levothyroxine | Linaclotide | | Sitagliptin | |
| | | Spironolactone | Losartan | Loratadine | | Trazodone | |
| | | Trazodone | Nitroglycerin | Losartan | | | |
| | | Valsartan | Pantoprazole | Metformin | | | |
| | | | Pioglitazone | Ondansetron | | | |
| | | | Tizanidine | Polyethylene Glycol | | | |
| | | | Topiramate | Sumatriptan | | | |
| | | | Trazodone | Trazodone | | | |
| | | | Valbenazine | Zofran | | | |
| Outcome | Non-Responder | Non-Responder | Non-Responder | Responder | Responder | Responder | Responder |
| % Improvement | 15.6 | 20.5 | 7.4 | 75.6 | 76.9 | 66.6 | 53.8 |

Unless defined otherwise, all technical and scientific terms used herein have the same meanings as commonly understood by one of skill in the art to which the disclosure belongs. Publications cited herein and the materials for which they are cited are specifically incorporated by reference.

Those skilled in the art will recognize or be able to ascertain using no more than routine experimentation, many equivalents to the specific embodiments of the disclosure. Such equivalents are intended to be encompassed by the following claims.

We claim:

1. A method for treating or preventing one or more symptoms associated with reduced gastrointestinal motility in a subject in need thereof, comprising electromagnetic stimulation of the thoracic sympathetic and/or parasympathetic nerves of the subject.

2. The method of claim 1, wherein the electromagnetic stimulation of the thoracic sympathetic and/or parasympathetic nerves comprises administering to one or more anatomical location(s) of the subject electromagnetic stimuli having an intensity between about 100% and about 150%, inclusive, of the minimal intensity of electromagnetic stimulation required to achieve a motor evoked response (MEP) value in the gastrointestinal (GI) tract of the subject,
optionally further comprising, prior to electromagnetic stimulation of the thoracic sympathetic and/or parasympathetic nerves of the subject, mapping of the thoracic regions of the subject,
wherein the mapping comprises identifying one or more preferred and/or optimal anatomical location(s) and a minimal intensity of electromagnetic stimulation required to achieve a motor evoked response (MEP) value in the gastrointestinal (GI) tract of the subject.

3. The method of claim 2, wherein the one or more optimal anatomical location(s) within the thoracic regions of the subject comprises the left and right posterior mid-thoracic regions.

4. The method of claim 2, wherein the MEP value is between about 1 μV and about 50 μV, inclusive,
optionally wherein the MEP value is about 10 μV.

5. The method of claim 2, wherein the MEP is evoked in the upper rectus abdominis and/or external oblique muscles of the subject.

6. The method of claim 2, wherein the method comprises mapping of the thoracic regions of the subject, and
wherein the mapping comprises electromagnetic stimulation applied to the subject through an electromagnetic coil,
optionally wherein the electromagnetic coil comprises a single-pulse circular 90 mm electromagnetic coil.

7. The method of claim 2, wherein the electromagnetic stimulation of the thoracic sympathetic and/or parasympathetic nerves comprises electromagnetic stimuli applied to the subject through an electromagnetic coil,
optionally wherein the electromagnetic coil comprises a 70 mm double air film self-cooling electromagnetic coil.

8. The method of claim 7, wherein the electromagnetic coil is held in place by a static coil fixator device throughout administration of the electromagnetic stimulation.

9. The method of claim 2, wherein the electromagnetic stimulation of the thoracic sympathetic and/or parasympathetic nerves comprises from about 100 to about 100,000 stimulations, inclusive, per anatomical location or from about 100 to about 100,000 stimulations, inclusive, per anatomical location or from about 1,000 to about 20,000 stimulations, inclusive, per anatomical location or from about 10,000 to about 15,000 stimulations, inclusive per anatomical location or wherein the electromagnetic stimuli comprises about 12,000 stimulations per anatomical location.

10. The method of claim 2, wherein the electromagnetic stimulation of the thoracic sympathetic and/or parasympathetic nerves comprises a frequency of between about 0.1 Hz and about 50 Hz, inclusive, or between about 0.5 Hz and about 5 Hz, inclusive, or
wherein the electromagnetic stimuli comprises a frequency of about 1 Hz.

11. The method of claim 2, wherein the electromagnetic stimulation of the thoracic sympathetic and/or parasympathetic nerves comprises electromagnetic stimuli administered to an anatomical location of the subject in two or more groups=,
wherein each group comprises less than the total number of stimulations, and
wherein the two or more groups are separated in time by a rest period.

12. The method of claim 11 wherein each group of stimuli comprises between about 10 and about 1,000 stimulations, inclusive, or between about 100 and about 500 stimulations, inclusive, or
wherein each group comprises about 300 stimulations.

13. The method of claim 11 wherein the rest period is between about 10 seconds and about 30 minutes, inclusive, or between about one minute and about ten minutes, inclusive, or
wherein the rest period is about five minutes.

14. The method of claim 2, further comprising repeating the electromagnetic stimuli of the thoracic sympathetic and/or parasympathetic nerves of the subject once or more than once,
optionally wherein the electromagnetic stimulation of the thoracic sympathetic and/or parasympathetic nerves of the subject is repeated at a regular interval.

15. The method of claim 14, wherein repeating the electromagnetic stimuli comprises
(i) mapping of the thoracic regions of the subject; and
(ii) administering to the one or more anatomical location(s) identified in the mapping electromagnetic stimuli having an intensity between about 100% and about 150%, inclusive, of the minimal intensity identified in the mapping.

16. The method of claim 14, wherein the interval is selected from the group consisting of one hour, 12 hours, one day, two days, one week, two weeks, three weeks, one month, two months, six months and one year.

17. The method of claim 14, wherein the electromagnetic stimuli are administered to a subject from two to twelve times a day, inclusive, for a period of five consecutive days.

18. The method of claim 2, wherein the method is effective to treat or prevent one or more symptoms selected from the group consisting of anal sphincter dysfunction, nausea, vomiting, retching, early satiety, postprandial fullness, bloating, loss of appetite, weight loss, abdominal distension and upper abdominal pain.

19. The method of claim 2, wherein the method is effective to treat or prevent one or more symptoms identified in the ANMS Gastroparesis Cardinal Symptom Index (ANMS GCSI-DD),
optionally wherein the method is effective to improve one or more of the symptoms identified in the ANMS GCSI-DD by one or more points,
wherein the points are defined as 0=none, 1=mild, 2=moderate, 3=severe, and 4=very severe.
wherein the subject does not have a metal implant or prosthesis.

20. A method to treat or prevent efferent spinogastric neuropathy and/or afferent gastro-cortical neuropathy in a subject comprising administering to the upper rectus abdominis and/or external oblique muscles of the subject electromagnetic stimuli having an intensity between about 100% and about 150%, inclusive, of the minimal intensity of electromagnetic stimulation
(i) required to achieve a motor evoked response (MEP) of 10 μV in the upper rectus abdominis and/or external oblique muscles of the subject, or
(ii) calculated as a graded stimulus based on body mass index (BMI) of the subject,
optionally wherein the method increases gastric contractility in the subject.
